(19) United States
(12) Patent Application Publication
(10) Pub. No.: US 2019/0197438 A1
(43) Pub. Date: Jun. 27, 2019

(54) SYSTEM AND METHOD FOR OPTIMALLY ROUTING AMBULANCES AND OTHER VEHICLES IN-ROUTE TO HOSPITALS

(71) Applicant: AT&T Intellectual Property I, L.P, Atlanta, GA (US)

(72) Inventors: Sheldon Meredith, Roswell, GA (US); William Cottrill, Canton, GA (US); Brandon Hilliard, Canton, GA (US)

(21) Appl. No.: 15/852,592

(22) Filed: Dec. 22, 2017

(51) Int. Cl. G06Q 10/04; G08G 1/00; G16H 40/20; G16H 10/60; G06Q 50/30

(52) U.S. Cl. CPC G06Q 10/047; G08G 1/202; G06Q 50/30; G16H 10/60; G16H 40/20

(57) ABSTRACT

A method for routing a patient to a care center includes receiving patient data and patient location information, identifying at least one care center, determining a location of at least one ambulatory vehicle, correlating the patient location with the location of the at least one ambulatory vehicle, determining a first routing instructions to the at least care center and transmitting the first routing instructions to the at least one ambulatory vehicle.

### TECHNICAL FIELD

This disclosure relates generally to using a mobile network for routing vehicles, and more specifically, to a system and method for determining an optimal route to transport patients to hospitals or urgent care centers in an emergency.

### BACKGROUND

In an emergency, the time required to transport an injured person to a hospital is critical and often could be the difference between life and death. Emergency response vehicles are often equipped with equipment and personnel which may be critical to saving lives. This means bringing the injured and the ambulance together may be a critical step on the way to a hospital or urgent care center. Distance or traffic congestion may impede efficient travel which is exacerbated if first an emergency vehicle may need to get to the injured and then proceed to a hospital. Even if the injured can be driven by another person to a hospital or urgent care center, distance and traffic may still cause life-threatening delays, compounded by the lack of professional care enroute to the hospital or urgent care center.

A use case highlights problems with the current state of the art. Consider the case where the injured is ten miles from a hospital and the ambulance is half way between the injured and the hospital. For the ambulance to transport the patient, it must travel 15 miles for the patient to arrive at the hospital. If a passerby transported the patient to the hospital, the vehicle would have to travel 10 miles, but that 10 miles is without the skill, expertise and equipment of first responders in the ambulance. So, it may be that the fastest way to get to the care center is for the driver to just drive to the closest one, but they may never meet up with the ambulance. Yes, the injured may have arrived at the hospital in a shorter amount of time, but suffered due to the lack of skilled care on the way there. There may also be the case wherein the route to the closest hospital is heavily congested or has road repairs and thus the second closest hospital may be optimal. It may be that the closest ambulance also has heavy traffic congestion or road repairs and thus the second closest ambulance may be optimal.

There is a need for a system and method to determine the quickest and most efficient route to transport a patient to a hospital or urgent care facility.

### SUMMARY

The present disclosure is directed to a system for routing a patient to a care center that includes an input-output interface, and a processor coupled to the input-output interface and wherein the processor is coupled to a memory, the memory having stored thereon executable instructions that when executed by the processor cause the processor to effectuate operations including receiving patient data and patient location information, identifying at least one care center, determining a location of at least one ambulatory vehicle, correlating the patient location with the location of the at least one ambulatory vehicle, determining a first routing instructions to the at least care center, and transmitting the first routing instructions to the at least one ambulatory vehicle. The operations may further include transmitting a second routing instruction to a non-emergency vehicle transporting the patient and may also include determining a waypoint for a meeting between a non-emergency vehicle transporting the patient and the at least one ambulatory vehicle, determining a second routing instruction from the non-emergency vehicle to the waypoint and transmitting the second routing instruction to the non-emergency vehicle and wherein the first routing instructions comprise a route to the waypoint. The first routing instructions may include routing to the location and then routing to the care center and wherein the routing to the waypoint and the routing to the care center are weighted in determining the routing. The operations may further include monitoring a chosen route and wherein the first routing instructions or the second routing instructions are updated based on the monitoring step. The operations may further include causing a voice connection to be established between the non-emergency vehicle and the ambulatory vehicle.

The present disclosure is also directed to an apparatus including an input-output interface, a processor coupled to the input-output interface and wherein the processor is coupled to a memory, the memory having stored thereon executable instructions that when executed by the processor cause the processor to effectuate operations including receiving information about a patient wherein the information comprises patient data and patient location, determining locations of at least two ambulatory vehicles and a care center available to treat the patient, weighting a route from each of the locations of the at least two ambulatory vehicles to the patient and then from the patient to the care center, calculating a weighted first transport time for a first one of the at least two ambulatory vehicles to travel to the patient and then from the patient to the care centers and a weighted second transport time for another of the at least two ambulatory vehicles to travel to the patient and then from the patient to the care center, and selecting the shorter of the weighted first transport time and the weighted second transport time. The calculating step may include delays attributable to at least one of traffic, construction, and special events and the patient data may include medical history data associated with the patient a list of possible injuries associated with the patient. The operations may further include transmitting a first route to one of the at least two ambulatory vehicles based on the selecting step and may further include monitoring the first route, adjusting the first route to create an adjusted route if delays are encountered along the first route and transmitting the adjusted route to the one of the at least two ambulatory vehicles. In an aspect, the operations may further include determining a waypoint for a meeting between a non-emergency vehicle transporting the patient and one of the at least two ambulatory vehicles, determining a second routing instruction from the non-emergency vehicle to the waypoint and transmitting the second routing instruction to the non-emergency vehicle and wherein the first route is a route to the waypoint. The operations may further include monitoring the first route, adjusting the first route to create an adjusted route if delays are encountered along the first route and transmitting the adjusted route to the one of the at least two ambulatory vehicles. In an aspect, the operations may further include establishing communication between the non-emergency vehicle transporting the patient and the one of the at least two ambulatory vehicles.

The present disclosure is also directed to a method including receiving patient information where the patient information includes information about injuries, determining a severity index based on the patient information, receiving patient location information, accessing a location of an ambulatory vehicle, selecting a care center based on the severity index, the patient location information, and the ambulatory vehicle location, and transmitting a first route to the emergency vehicle wherein the route includes a route to the patient location and a route to the care center from the patient location. The method may further include wherein the patient is in a non-emergency vehicle and the steps further include determining a waypoint for meeting between the non-emergency vehicle and the emergency vehicle, transmitting a second route to the non-emergency vehicle and the waypoint and wherein the first route includes a route to the waypoint instead of a route to the patient location. The first route and the second route may be optimized based on total travel time for the patient to reach the care center and the severity index. The method may further include first route and second route are monitored and the waypoint is adjusted based on one of traffic, construction and special events.

### BRIEF DESCRIPTION OF THE DRAWINGS

In the following description, for purposes of explanation, numerous specific details are set forth in order to provide an understanding of the variations in implementing the disclosed technology. However, the instant disclosure may take many different forms and should not be construed as limited to the examples set forth herein. Where practical, like numbers refer to like elements throughout.

FIG. 1 is a representation of an exemplary operating environment for a Public Safety Access Point (PSAP) in communication with first responder vehicles and an emergency application in a connected vehicle or smartphone.

FIG. 2 is a representation of an exemplary operating environment showing a connected vehicle, urgent care centers and first responder vehicles superimposed on a city grid.

FIG. 3 is a functional block diagram of an exemplary mobile emergency application which may operate on a client such as a smartphone, tablet, portable computer or a connected vehicle.

FIG. 4 is an exemplary flow diagram of a process which may be performed by a PSAP server.

FIG. 5 is an exemplary flow diagram of a process which may be performed by a client application in accordance with the present disclosure.

FIG. 6 is an exemplary flow diagram of a process for determining the most optimal route for emergency transport of a patient.

### DETAILED DESCRIPTION

Overview.

As detailed herein, the present disclosure is directed to a system and method for providing the most efficient route and vehicle to transport a patient to a hospital using limited information about the injured person or persons resulting from a 911 call to a public safety answering point (PSAP). A mobile emergency application operating on a mobile device or a connected vehicle may be used. For the purposes of this disclosure, the term “mobile emergency application” is used interchangeably with “client application” unless otherwise specified. Functionally, the PSAP may remotely provide information to the mobile emergency application, where such information results from the PSAP operator manually entering information derived from the voice interaction with the caller or from interactions with a PSAP server which would automate communications with the mobile emergency application. Alternatively, the mobile emergency application may detect critical keywords or phrases like dying, bleeding, concussion, or the like to determine the medical treatment most likely needed for the patient and thereby determine the medical facility that would be best able to attend to the patient. In another embodiment, the caller may manually interact with the mobile emergency application, either by answering questions or entering information free form or from a predetermined menu.

In an aspect, the mobile device and/or connected-car may provide the PSAP with GPS-quality location information. Likewise the ambulatory vehicles may also provide location information to PSAPs via a wireless data connection. At the PSAP, a software application running on server may interpret the characteristics of the emergency event, which may, for example, include codes for a heart attack, severe cut, eye injury, burns, etc. and perhaps a severity index, in conjunction with the locations of the ambulatory vehicles, the location of the injured person/persons, hospitals or urgent care centers with the appropriate emergency services available, and other information about the road network and road conditions to automatically provide routing information to transport the patient to the hospital or urgent care facility. The routing information may be used by both ambulatory vehicles and a vehicle at the location of the injured person to route the two vehicles into close proximity in-route to the hospital or urgent care facility. This permits a handoff of the injured patient to the ambulance personnel prior to arrival at a hospital or urgent care center such that paramedics experts may render life-saving skills using emergency equipment to stabilize and treat the patient and begin the coordination with the hospital or urgent care center. Hospitals or urgent care centers will be referred to broadly herein as care centers.

System Environment.

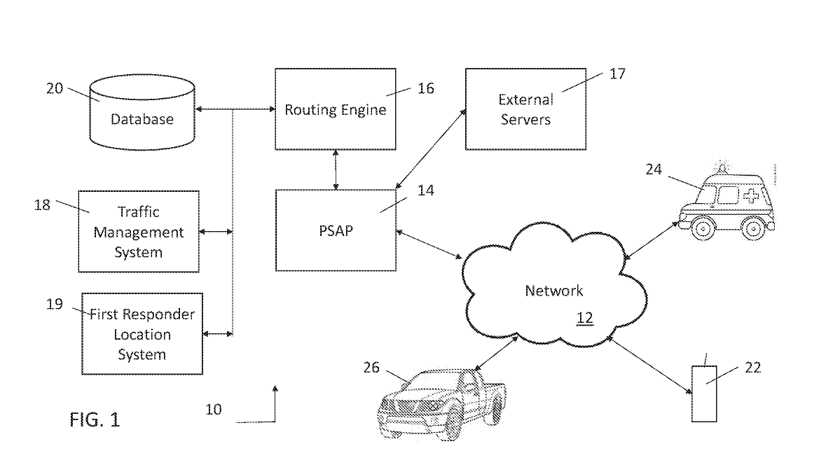

With reference to FIG. 1, there is shown a system 10 in which the present disclosure may operate. Central to the system is network 12, which may be a combination of wireless and/or wired network communication systems. The network 12 may include components of the Public Switched Telephone Network (PSTN) as well as wireless network systems including 3G, 4G/LTE, 5G, WiFi, WiMAX, and any other wireless network communications system now known or to be developed in the future. The network 12 itself and voice and data communications across the network 12 is known by those skilled in the art

In communication with the network 12 is a PSAP gateway 14. PSAP gateway 14 may interact with a routing engine 16 which in turn is interactive with a traffic management system 18, a first responder location system 19 and database 20. The database 20 may include information on hospital locations and capabilities, maps of roads and other transportation infrastructure, an identification of first responder ambulatory vehicles and their capabilities and any other data which may be relevant to the systems and methods of the present disclosure.

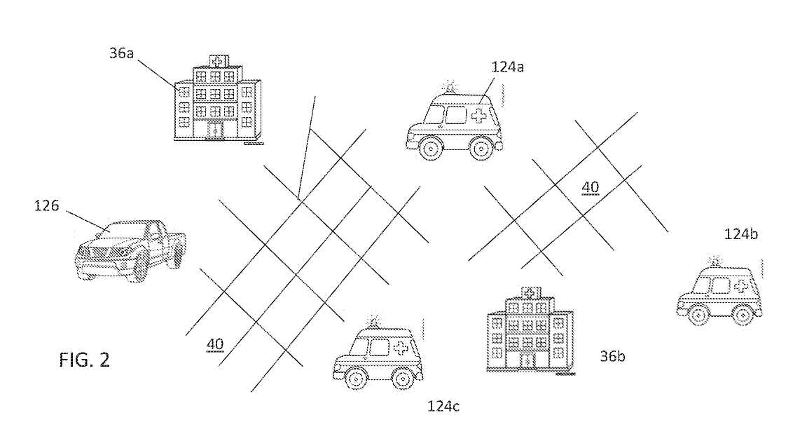

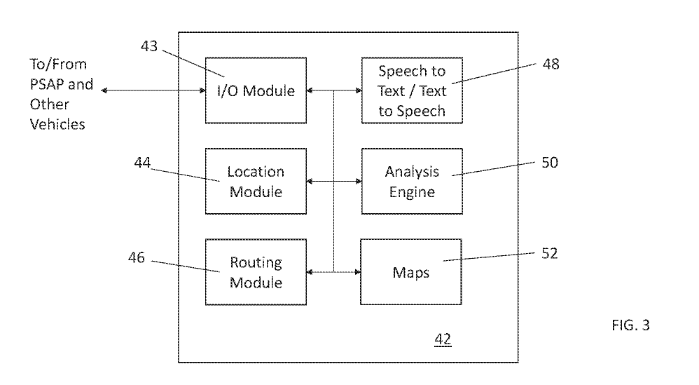

The first responder location system 19 is an option which would track the location of first responder ambulatory vehicles 24. Such ambulatory vehicles are also shown as 124a, 124b and 124c in FIG. 2. The first responder location system may keep a record of dispatches, routes and destinations for one or more ambulatory vehicles that is updated continuously or periodically such that the PSAP gateway 14 may have real-time or near real-time access to the location of first responder ambulatory vehicle 24. The PSAP server 14 may automatically request an update of the locations of first responder vehicles periodically, automatically receive push notifications form the first responder location system 19 periodically, or specifically request the location of one or more first responder ambulatory vehicles on command or as a result of an inquiry from a mobile application shown as 42 in FIG. 3. The mobile application will be discussed in more detail with reference to FIG. 3 below.

There is also shown a traffic management system 18. The traffic management system 18 may include real time status relating to traffic, accidents, road construction, special events, and any other information that may affect traffic patterns or traffic speeds. The traffic information may be correlated internally at a PSAP location or periodically or a-periodically retrieved from external servers 17. The traffic management system 18 may interact with street map data stored in database 20 which may, for example, include roads, street lights, stop signs, bus routes, commuter patterns or any other data relating to roads or road conditions. The traffic management system 18 may receive feeds from state, local or private sources in order to contain the most up to date traffic and road conditions.

Database 20 may also include location information, capacities and capabilities of care centers including hospitals, emergency rooms, doctors, heliports, nursing homes, temporary or permanent shelters, trauma units or any other location wherein a patient may obtain medical care. For each such care location, there may be stored the capabilities or specialties available at each care location, including for example, trauma centers, surgical capabilities, first aid, orthopedics, x-rays, burn specialists or any other type of medical specialty or capability.

Continuing with the description of the operating environment in FIG. 1, the PSAP server 14 may be in communication with first responder ambulatory vehicle 24 through network 12 which may include voice and data capabilities. PSAP server 14 may communicate with a device, such as a laptop, tablet, or other communications device within the first responder vehicle 24. Such communications may include dispatch information, patient data, patient location, and other information, including routing information to be discussed in greater detail below. The PSAP server 14 may also be in communication with a mobile emergency application 42 running on user device 22 or incorporated into a connected vehicle 26. For exemplary purposes only, the user device 22 and connected vehicle 26 are used as an example of a client device hosting a mobile emergency application 42. Those skilled in the art will understand that a connected vehicle 26 may include monitoring sensors and wireless communication links, including bi-directional voice and data communications, useful for a variety of purposes. The user device 22 may be within the connected vehicle 26 or serve as the connectivity for any vehicle that may be involved in an accident or the transport of a patient to a care center. For the purposes of a non-limiting example only, connected vehicle 26 will be used to describe a vehicle that may originally transport a patient to either a care facility shown as 36a and 36b in FIG. 2 a meeting point with a first responder ambulatory vehicle 24, or a non-transport vehicle which simply provides the location of an injured person. For the purposes of a non-limiting example only, the location of and communication with an injured person or a person transporting the injured person will be through the connected vehicle 26 location and communication systems and/or the user device 22.

The PSAP server 14 may also communicate with external servers 17 to obtain additional information. Such additional information may include, but is not limited to, real time traffic reports and conditions, weather conditions, road construction schedules, special event schedules, natural disasters or other emergency situations, social media, patient health records, or any other externally generated information which may be useful to the PSAP, first responders, or care facilities in treating patient injuries. The PSAP server 14 may control or otherwise facilitate communications to and from the user device 22 or connected vehicle 26, a first responder ambulatory vehicle 24, PSAP personnel, and care facilities 36a 36b. Also shown in FIG. 1 is a routing engine 16. The routing engine 16 may receive as inputs such as the type and number of patients and their injuries, patient location, care facility locations 36a, 36b, first responder ambulatory vehicle 24 locations, traffic information, street maps, and any other information in order to provide routing instructions to the first responder ambulatory vehicle 24 and the connected vehicle 26 or smartphone 22. The routing function will be described in more detail below.

The operating environment is further described with reference to FIG. 2 which shows care facilities 36a, 36b, connected vehicle 126, and first responder vehicles 124a, 124b, 124c laid out on a grid 40 comprising roads, bridges, parking lots, public transportation routes and the like. Each care facility 36a, 36b may have the same or different general or specific care specialties, emergency rooms, capacities in terms of number of beds or number of patients that can be triaged, number of doctors and support staff, insurance contracts, ambulatory contracts or the like. As such, in addition to location considerations, the PSAP personnel or PSAP server 14 may direct a patient to a particular care facility 36a, 36b based on the capabilities of the care facility 36a, 36b and the needs of the patient.

Emergency Application.

With reference to FIG. 3, there is shown an exemplary mobile emergency application 42 (also referred to as client application herein) that may reside in a connected vehicle 26 or a user device 22. The client application 42 may include a input/output processor 43 to facilitate communications between the connected vehicle 26 or user device 22 and a PSAP server 14, occupants of the connected vehicle 26 or users of the user device 22, with other vehicles or care facilities 36a, 26b. In an aspect, the input/output processor 43 may support voice and data wireless communication over 3G, UMTS, 4G/LTE, 5G, Wi-Fi, Wi-Max or any other wireless network. The input/output processer 43 may also interface with a keyboard, an interactive touch-screen display, a regular display, a microphone, a speaker, a mouse, touchpad, or any other input/output device.

Also included in the client application 42 may be a mapping function 52 which may, for example, provide street maps, points of interest, or other mapping functions. A geo-location system 44 may be included to determine the location of the connected vehicle 26 or user device 22. The geo-location system module 44 may use GPS, a-GPS, time delay of arrival, triangulation, or any other method of determining the location of the connected vehicle 26 or user device 22. There may also be a routing module 46 which may operate independently of any instructions from the PSAP server 14 or in conjunction with instructions received from the PSAP server 14 to provide directions for travel. For example, independent operation may include providing routing to a destination by inputting the destination and receiving the current location from the geo-location system 44 to determine possible routes to the destination as is known in the art. Alternatively, the PSAP server 14 may provide the optimal routing or one or more waypoints to the connected vehicle 26 or user device 22.

It may be useful for the connected vehicle 26 or the user device 22 to operate in a hands free mode, therefore a text to speech and/or a speech to text system 48 may be included in the client application 42. Using the text to speech function, a user may receive inputs from the PSAP server 14 with respect to routing instructions and, in addition to a visual display showing the routing information, the driver of the connected vehicle 26 or user of the user device 22 may also hear the destination or waypoint and be verbally advised as to the turn by turn routing instructions. Conversely, speech to text functionality may be included in order for the driver of the connected vehicle 26 or user of the user device 22 to communicate via data messaging with a PSAP server 14 or PSAP personnel in a hands-free environment.

Also included in the client emergency application may be an analysis module 50. The analysis module 50 may, for example, analyze symptoms, the circumstances surrounding an accident, and other data from the scene and/or the vehicle and thereafter provide preliminary diagnosis, either alone or in combination with another program operating on the PSAP server 14. For example, a non-patient at the scene of an accident may input symptoms displayed by the patient, i.e, head wounds, bleeding, broken bones, back pain, burns, or any other symptoms, and a description of the accident type and site, i.e., head on collision, side-swipe, car fire, explosion, or other descriptors of the accident site, and then provide a preliminary diagnosis to the PSAP server 14 for relaying to a care center 36a, 36b. The input may be via voice, text, or video. Alternatively, the analysis module 50 may collect key words input from an accident scene uttered by a user and transmit those key words to the PSAP server 14 for further analysis.

In an aspect, the analysis engine 50 may include an artificial intelligence (AI) element incorporating deep learning components, thereby enabling the AI engine to store a plurality of records and “learn” from interactions between the PSAP server 14, the first responder ambulatory vehicles 24 and care facilities 36a, 36b. The AI engine may, for example, be both predictive and prescriptive in its analytics, thereby facilitating timely forward-predictive conditions and response scenarios.

In an aspect, the AI-based predictive and prescriptive analytics elements may proactively and dynamically respond to traffic conditions and events or other outside forces such as weather or local or regional events or disasters that could impact road conditions and thereby affect transport times, care center capacities and capabilities, and individual or mass casualties, which if unattended, may adversely impact response time and transportation to the appropriate urgent care facility 36a, 36b. In this manner, the AI engine may dynamically interface with PSAP server 14 and external servers 17 in near-real time to proactively modify routing determinations, including re-routing of the transport between the first responders ambulatory vehicles and individual transport vehicles such as the connected vehicle 126.

PSAP server 14 and the client application 42 may each be implemented as a general purpose computer programmed to provide the functions set forth above, and as such, each may have a CPU function and a memory for storing executable instructions thereon.

It will be understood by those skilled in the art that some or all of the analysis functions described above with respect to the client application 42 may be performed by an application running on the PSAP server 14 or any other server or processor in communication with PSAP server 14. In either case, as much information as reasonably practical under the circumstances that is conveyed from an accident or incident site to the PSAP server 14 will assist in defining the optimal route for transporting a patient to the hospital.

Methods of Use.

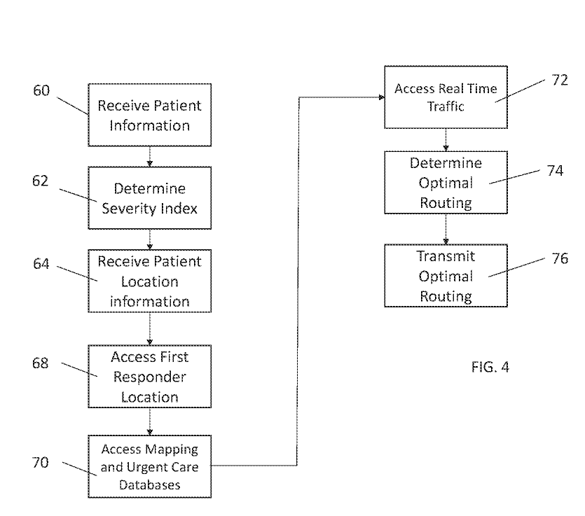

With reference to FIG. 4, there is shown a flow chart of the system and method of the present disclosure in operation from the perspective of a PSAP after receipt of an emergency call. At 60, patient information is received. Such patient information may, for example, include the patient's age, gender, height, weight, (or approximations of the foregoing) or any other general information about the patient. Additional patient information may include the key words describing the suspected injuries or the preliminary analysis of the patient injuries as described above. Patient information may also include patient history, which may, for example, be stored or accessed by client application 42 and transmitted to the PSAP server 14. In the event of a multiple injury situation, patient information may also include the number of patients and individual information about each of those patients and any common facts associated with the multiple injuries.

At 62, the PSAP may determine the severity index of the patient based on the received patient information. For example, the severity index may designate injuries as being on the continuum from minor to severe to life threatening, depending on the symptoms and preliminary analysis. For example, in a head-on accident, there may be symptoms of head injuries that include bleeding and confusing behavior, indicating that the patient may have suffered a concussive event. The severity index may be communicated via voice, video or text or some other messaging application to the PSAP server 14. At 64, patient location information is received. The patient location information may include whether the patient has access to transportation which may, for example, be a connected vehicle 126 or any other transport vehicle. If that is the case, the most optimum route may include having an emergency vehicle 124a, 124b, 124c meeting the connected vehicle 126 or other transport vehicle at a location on the path towards the care center but not at the accident zone. If there is no transportation available for the patient, then one of a plurality the emergency vehicle 124a, 124b, 124c may be directly dispatched to the accident zone.

Once the patient location information is received, the first responder location is accessed at 68. This may include accessing database 20 or querying one or more first responder ambulatory vehicles 124a, 124b, 124c to receive their respective locations with respect to grid 40. At 70, urgent care locations and capabilities are determined, which determination may, for example, include a query to database 20. Additionally at 70, mapping functionality may also be accessed from database 20. It will be understood by those skilled in the art that the urgent care and mapping data do not need to be part of the same database 20. The mapping functionality may be accessed from a third party provider, for example, Google® Maps. The mapping functionality may also include areas of road construction or other circumstances which may impact traffic flow.

At 72, real time traffic is accessed. Real time traffic may also include near real time traffic and may, for example, be retrieved from an external commercial traffic monitoring server or maintained locally at the PSAP by reception of traffic feeds from state and local authorities or traffic monitoring firms, including radio stations. Real time traffic may include not only congestion, but also traffic signal outages, road construction, events such as a parade, or any other information that may positively or adversely affect traffic flow. At 74, optimal routing is determined by a routing engine 16. Optimal routing may include determining a meeting location for the connected vehicle 126 or other transport vehicle tasked with transporting a patient from an accident site to a rendezvous site and a designated first responder ambulatory vehicle selected from a plurality of first responder ambulatory vehicles (i.e., first responder ambulatory vehicle 124a). If a rendezvous place is determined to be desired, then optimal routing may include the optimal routing to the rendezvous point for both the connected vehicle 126 or other transport vehicle and the first responder ambulatory vehicle 124a. The optimal routing information is transmitted at 76 to both the connected vehicle 126 and the first responder ambulatory vehicle 124a.

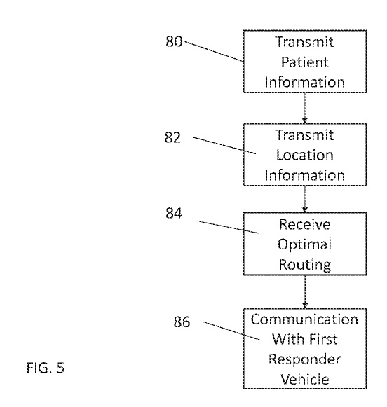

FIG. 5 shows an exemplary flow diagram from the perspective of the connected vehicle 126 or client application 42. At 80, the patient information is transmitted to the PSAP server 14, The patient information may include pre-stored data relating to the patient such as patient identification including age, gender, address, general health condition, medical history, or other patient information. Some of such patient information may be prestored on the client application or accessed from an external database. Other patient information may be entered through the I/O module 43. At 82, the location of the patient is transmitted, including whether the patient has access to local, non-emergency transportation through a connected vehicle 126 or other transport vehicle. At 84, the client application 42 receives the optimal routing information from the PSAP server 14. At 86, communication is established between the client application 42 and the first responder ambulatory vehicle, for example, first responder ambulatory vehicle 124a,

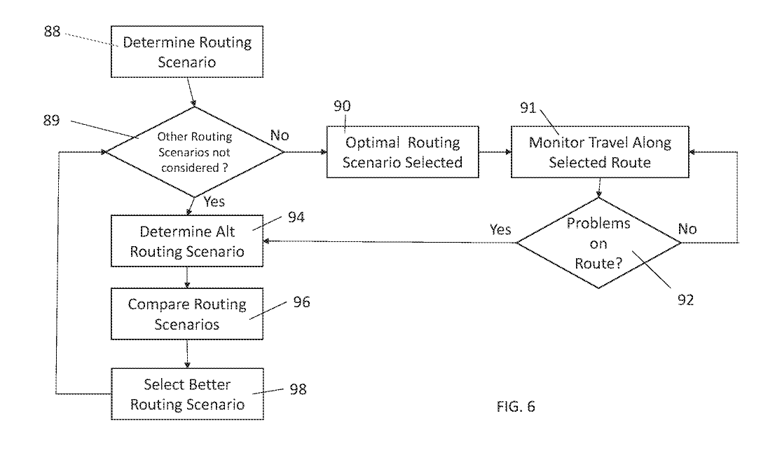

FIG. 6 shows an exemplary flow diagram of a program to illustrate how an optimal routing scenario may be determined at 88. It will be understood that this flow diagram is exemplary only and there are other methods within the scope of the present disclosure and appended claims for determining an optimal routing scenario. At 89, an inquiry is made as to whether there are alternative routing scenarios that have not yet been considered. If there are such alternative routing scenarios available, such alternative routing scenarios are determined at 94. The alternative routing scenarios are then compared with the actual routing scenario at 96 and the better routing scenario is selected at 98. The process repeats at 89 to determine whether still other alternative routing scenarios not yet considered exist until the optimal routing scenario is selected. Once it is determined that there are no other alternative scenarios not considered, then the process continues at 90 where the optimal routing scenario is selected. At 91, travel on the selected route is monitored. At 92, a query as to whether there are problems encountered on the route have been discovered. If not, the monitoring continues at 91 and a follow-up inquiry at 92. If there are problems encountered, the process continues at 94 to determine an alternative routing scenario as described above. In this manner, any time a problem on the designated route is discovered through a monitoring function, the system may continually calculate travel times and distances to ensure that the optimal route under the circumstances is being followed.

Exemplary Use Cases.

With reference to FIG. 2, the present disclosure may be useful in the case in which someone has lost a limb and a lot of blood and now has a tourniquet and is in a transport vehicle 126. There are three first responder ambulatory vehicles 124a, 124b, 124c in proximity and available to assist. There are two care centers 36a, 36b.

Assume, that f transport vehicle 126 is ten miles from care center 36a and 12 miles from care center 36b. The probably injury and severity index is identified by the client application 42 and transmitted to PSAP server 14. PSAP server 14 then queries database 20. The PSAP server 14 then calculates that connecting the injured patient with the ambulance is 70% of the challenge and the remaining 30% of the challenge is transporting the injured patient to one of the care centers 36a, 36b. The routing engine 16 considers all possible first responder ambulatory vehicles 124a, 124b and 124c and all care centers 36a, 36b. Each possibility may then be tested. A first ambulatory vehicle, i.e. ambulatory vehicle 124a, and a care center 36a may be selected as the optimal combination of ambulatory vehicle and care center.

At that point, the most efficient route to the care center 36a may be determined with consideration given to diversions in time, which may, for example, consist of the patient simply waiting somewhere for the ambulance, and to road conditions that may impact the most optimal route to engage the ambulatory vehicle 124a.

In an aspect, two travel times for each test location and time delay are calculated, namely, the time to get to the ambulance and the time to ultimately get to the care center 36a (including some transfer time to the ambulatory vehicle 124a). In this example, these times may be weighted by factors 0.7 and 0.3 respectively, with the results then summed. As such, the travel time includes both street and current traffic information. This process is repeated for a range of time and route diversions, including diversions to different care centers, for example, care center 36b. The route and rendezvous point with the best weighted time is then selected.

Automated software routines in the PSAP server 14 or other connected computer system that know the accurate locations of all vehicles and hospitals along with road networks and traffic conditions are very quickly able to determine the best location for a specific ambulatory vehicle to meet with a transport vehicle in-route to the optimal care center for a minimum weighted delivery time. Since the PSAP server 14 may be wirelessly connected to the connected vehicle 126, transport vehicle, or mobile device 22, along with the chosen ambulatory vehicle 124a, 124b, or 124c, these vehicles are routed towards each other in-route to the chosen care center and such routing information is continuously updated to account for travel deviations from predictions. Information about connected vehicle 126 or other transport vehicle, for example, the color, make, model, and license plate may be automatically uploaded from the transport vehicle to the PSAP server 14 and shared with the ambulatory vehicle 124a, 124b, 124c so the ambulance personnel can identify the transport vehicle. Further, a voice link may be established between connected car 126 or other transport vehicle and the chosen ambulatory vehicle 124a, 124b, or 124c to allow direct communication between the two when they are in proximity to each other.

While examples of a system and method in which patients and deployed emergency ambulatory vehicles can be directed to a care facility in an optimal manner have been described in connection with various computing devices/processors, the underlying concepts may be applied to any computing device, processor, or system capable of facilitating a telecommunications system. The various techniques described herein may be implemented in connection with hardware or software or, where appropriate, with a combination of both. Thus, the methods and devices may take the form of program code (i.e., instructions) embodied in concrete, tangible, storage media having a concrete, tangible, physical structure. Examples of tangible storage media include floppy diskettes, Compact Disc-Read-Only Memory devices (CD-ROMs), Digital Versatile Discs, or, Digital Video Discs (DVDs), hard drives, or any other tangible machine-readable storage medium (computer-readable storage medium). Thus, a computer-readable storage medium is not a signal. A computer-readable storage medium is not a transient signal. Further, a computer-readable storage medium is not a propagating signal. A computer-readable storage medium as described herein is an article of manufacture. When the program code is loaded into and executed by a machine, such as a computer, the machine becomes a device for telecommunications. In the case of program code execution on programmable computers, the computing device will generally include a processor, a storage medium readable by the processor (including volatile or nonvolatile memory or storage elements), at least one input device, and at least one output device. The program(s) can be implemented in assembly or machine language, if desired. The language can be a compiled or interpreted language, and may be combined with hardware implementations.

The methods and devices associated with the disclosure described herein also may be practiced via communications embodied in the form of program code that is transmitted over some transmission medium, such as over electrical wiring or cabling, through fiber optics, or via any other form of transmission, over the air (OTA), or firmware over the air (FOTA), wherein, when the program code is received and loaded into and executed by a machine, such as an Erasable Programmable Read-Only Memory (EPROM), a gate array, a programmable logic device (PLD), a client computer, or the like, the machine becomes an device for implementing telecommunications as described herein. When implemented on a general-purpose processor, the program code combines with the processor to provide a unique device that operates to invoke the functionality of the system described herein. Functions described as operating on a client device may be operating on a server and vice versa.

It will be apparent to those skilled in the art that various modifications and variations may be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system for routing a patient to a care center comprising:
An input-output interface; and
a processor coupled to the input-output interface and wherein the processor is coupled to a memory, the memory having stored thereon executable instructions that when executed by the processor cause the processor to effectuate operations comprising:
receiving patient data and patient location information;
identifying at least one care center;
determining a location of at least one ambulatory vehicle;
correlating the patient location with the location of the at least one ambulatory vehicle;
determining a first routing instructions to the at least care center; and
transmitting the first routing instructions to the at least one ambulatory vehicle.

2. The system of claim 1 wherein the steps further comprise:
Transmitting a second routing instruction to a non-emergency vehicle transporting the patient.

3. The system of claim 1 wherein the operations further comprise determining a waypoint for a meeting between a non-emergency vehicle transporting the patient and the at least one ambulatory vehicle, determining a second routing instruction from the non-emergency vehicle to the waypoint and transmitting the second routing instruction to the non-emergency vehicle and wherein the first routing instructions comprise a route to the waypoint.

4. The system of claim 3 wherein the first routing instructions include routing to the location and then routing to the care center.

5. The system of claim 3 wherein the routing to the waypoint and the routing to the care center are weighted in determining the routing.

6. The system of claim 3 wherein the operations further comprise monitoring a chosen route and wherein the first routing instructions or the second routing instructions are updated based on the monitoring step.

7. The system of claim 3 further comprising causing a voice connection to be established between the non-emergency vehicle and the ambulatory vehicle.

8. An apparatus comprising:
an input-output interface;
a processor coupled to the input-output interface and wherein the processor is coupled to a memory, the memory having stored thereon executable instructions that when executed by the processor cause the processor to effectuate operations comprising:
receiving information about a patient wherein the information comprises patient data and patient location;
determining locations of at least two ambulatory vehicles and a care center available to treat the patient;
weighting a route from each of the locations of the at least two ambulatory vehicles to the patient and then from the patient to the care center;
calculating a weighted first transport time for a first one of the at least two ambulatory vehicles to travel to the patient and then from the patient to the care centers and a weighted second transport time for another of the at least two ambulatory vehicles to travel to the patient and then from the patient to the care center; and
selecting the shorter of the weighted first transport time and the weighted second transport time.

9. The apparatus of claim 8 wherein the operations wherein the calculating step includes delays attributable to at least one of traffic, construction, and special events.

10. The apparatus of claim 8 wherein the patient data includes medical history data associated with the patient.

11. The apparatus of claim 8 wherein the patient data includes a list of possible injuries associated with the patient.

12. The apparatus of claim 8 wherein the operations further comprise transmitting a first route to one of the at least two ambulatory vehicles based on the selecting step.

13. The apparatus of claim 12 wherein the operations further comprise monitoring the first route, adjusting the first route to create an adjusted route if delays are encountered along the first route and transmitting the adjusted route to the one of the at least two ambulatory vehicles.

14. The apparatus of claim 12 wherein the operations further include determining a waypoint for a meeting between a non-emergency vehicle transporting the patient and one of the at least two ambulatory vehicles, determining a second routing instruction from the non-emergency vehicle to the waypoint and transmitting the second routing instruction to the non-emergency vehicle and wherein the first route is a route to the waypoint.

15. The apparatus of claim 14 wherein the operations further comprise monitoring the first route, adjusting the first route to create an adjusted route if delays are encountered along the first route and transmitting the adjusted route to the one of the at least two ambulatory vehicles.

16. The apparatus of claim 14 wherein the operations further comprise establishing communication between the non-emergency vehicle transporting the patient and the one of the at least two ambulatory vehicles.

17. A method comprising:
Receiving patient information where the patient information includes information about injuries;
Determining a severity index based on the patient information;
Receiving patient location information;
Accessing a location of an ambulatory vehicle;
Selecting a care center based on the severity index, the patient location information, and the first responder location;
Transmitting a first route to the ambulatory vehicle wherein the route includes a route to the patient location and a route to the care center from the patient location.

18. The method of claim 17 wherein the patient is in a non-emergency vehicle and the steps further comprise determining a waypoint for meeting between the non-emergency vehicle and the ambulatory vehicle, transmitting a second route to the non-emergency vehicle and the waypoint and wherein the first route includes a route to the waypoint instead of a route to the patient location.

19. The method of claim 18 wherein the first route and the second route are optimized based on total travel time for the patient to reach the care center and the severity index.

20. The method of claim 19 wherein the first route and second route are monitored and the waypoint is adjusted based on one of traffic, construction and special events.
